(19) United States
(12) Patent Application Publication
(10) Pub. No.: US 2018/0371492 A1
(43) Pub. Date: Dec. 27, 2018

(54) METHODS AND COMPOSITIONS RELATING TO ASSAYS FOR INHIBITORS OF ASSOCIATION BETWEEN ELK1 AND STEROID HORMONE RECEPTORS

(71) Applicant: Wayne State University, Detroit, MI (US)

(72) Inventor: Manohar Ratnam, Toledo, OH (US)

(21) Appl. No.: 16/019,218

(22) Filed: Jun. 26, 2018

Related U.S. Application Data
(60) Provisional application No. 62/524,891, filed on Jun. 26, 2017.

(51) Int. Cl. C12N 15/85; C12Q 1/6886; C12N 15/62; C12N 15/10

(52) U.S. Cl. CPC C12N 15/85; C12Q 1/6886; C12N 15/62; C12N 2015/859; C12Q 2600/106; C12Q 2600/136; C12N 15/1086

(57) ABSTRACT

Assay systems for identifying a compound characterized by anti-hormone receptor-dependent cancer activity are provided according to aspects of the present invention which include: I) a first recombinant cell including: 1) a reporter gene expression construct driven by a DNA binding domain recognition sequence; 2) an expression construct encoding a DNA binding domain and ELK1 lacking an ETS DNA binding domain; and 3) an expression construct encoding human androgen receptor (AR); and II) a second recombinant cell including: an androgen response element (ARE)-reporter gene expression construct including an ARE in operable linkage with a nucleic acid encoding a reporter; and an expression construct encoding AR, wherein in the presence of androgen, the AR enters the nucleus of the second recombinant cell and specifically binds to the ARE, thereby activating expression of the reporter gene in the second recombinant cell.

### REFERENCE TO RELATED APPLICATION

This application claims priority from U.S. Provisional Patent Application Ser. No. 62/524,891, filed Jun. 26, 2017, the entire content of which is incorporated herein by reference.

### FIELD OF THE INVENTION

General aspects are described relating to systems and methods for identifying compounds having activity against steroid hormone-receptor-dependent cancer. According to specific aspects, assay systems and methods of use for identifying compounds having activity against androgen-receptor-dependent cancer.

### BACKGROUND OF THE INVENTION

There is a continuing need for methods and compositions relating to hormone-receptor-dependent cancers, including systems and methods for identifying compounds having activity against steroid hormone-receptor-dependent cancer, such as androgen-receptor-dependent cancer.

### SUMMARY OF THE INVENTION

Assay systems for identifying a compound characterized by anti-hormone receptor-dependent cancer activity are provided according to aspects of the present invention which include: I) a first recombinant cell having a nucleus, the first recombinant cell including: 1) a reporter gene expression construct including a DNA binding domain recognition sequence in operable linkage with a nucleotide sequence encoding a reporter; 2) an expression construct encoding a fusion protein, the fusion protein including a DNA binding domain and ELK1 (ETS transcription factor (ELK1), transcript variant 1) lacking an ETS (E-twenty-six) DNA binding domain wherein the DNA binding domain is substituted for the ETS DNA binding domain of ELK1; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the first recombinant cell and specifically binds to ELK1 in the DNA binding domain-ELK1 fusion protein, thereby activating expression of the reporter gene in the first recombinant cell; and II) a second recombinant cell, the second recombinant cell including: an androgen response element (ARE)-reporter gene expression construct including an ARE in operable linkage with a nucleic acid encoding a reporter, and an expression construct encoding human androgen receptor (AR), wherein in the presence of androgen, the AR enters the nucleus of the second recombinant cell and specifically binds to the ARE, thereby activating expression of the reporter gene in the second recombinant cell. Optionally, the reporter gene expression construct; the expression construct encoding a fusion protein; the expression construct encoding AR, any two thereof, or all of these, is stably integrated in the genome of the first recombinant cell. Optionally, one or both of the ARE-reporter gene expression construct; and the expression construct encoding AR, is stably integrated in the genome of the second recombinant cell. Optionally, the reporter gene expression construct of the first recombinant cell and the reporter gene expression construct of the second recombinant cell both encode the same reporter. Optionally, the reporter gene expression construct of the first recombinant cell and the reporter gene expression construct of the second recombinant cell both encode firefly luciferase. Optionally, the reporter gene expression construct of the first recombinant cell and the reporter gene expression construct of the second recombinant cell encode different reporters.

Assay systems for identifying a compound characterized by anti-hormone receptor-dependent cancer activity are provided according to aspects of the present invention which include: I) a first recombinant cell having a nucleus, the first recombinant cell including: 1) a reporter gene expression construct including a DNA binding domain recognition sequence in operable linkage with a nucleotide sequence encoding a reporter, wherein the DNA binding domain recognition sequence includes a Gal4 DNA binding domain recognition sequence, or two or more contiguous Gal4 DNA binding domain recognition sequences; 2) an expression construct encoding a fusion protein, the fusion protein including a DNA binding domain and ELK1 lacking an ETS DNA binding domain, wherein the DNA binding domain is substituted for the ETS DNA binding domain of ELK1 and wherein the DNA binding domain of the fusion protein is a Gal4 DNA binding domain that specifically binds to the Gal4 DNA binding domain recognition sequence or to the two or more contiguous Gal4 DNA binding domain recognition sequences; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the first recombinant cell and specifically binds to ELK1 in the DNA binding domain-ELK1 fusion protein, thereby activating expression of the reporter gene in the first recombinant cell; and II) a second recombinant cell, the second recombinant cell including: an androgen response element (ARE)-reporter gene expression construct including an ARE in operable linkage with a nucleic acid encoding a reporter; and an expression construct encoding human androgen receptor (AR), wherein in the presence of androgen, the AR enters the nucleus of the second recombinant cell and specifically binds to the ARE, thereby activating expression of the reporter gene in the second recombinant cell. Optionally, the reporter gene expression construct; the expression construct encoding a fusion protein; the expression construct encoding AR, any two thereof, or all of these, is stably integrated in the genome of the first recombinant cell. Optionally, one or both of the ARE-reporter gene expression construct; and the expression construct encoding AR, is stably integrated in the genome of the second recombinant cell. Optionally, the reporter gene expression construct of the first recombinant cell and the reporter gene expression construct of the second recombinant cell both encode the same reporter. Optionally, the reporter gene expression construct of the first recombinant cell and the reporter gene expression construct of the second recombinant cell both encode firefly luciferase. Optionally, the reporter gene expression construct of the first recombinant cell and the reporter gene expression construct of the second recombinant cell encode different reporters.

An assay to identify a compound characterized by anti-hormone receptor-dependent cancer activity is provided according aspects of the present invention which include: providing I) a first recombinant cell having a nucleus, the first recombinant cell including: 1) a reporter gene expression construct including a DNA binding domain recognition sequence in operable linkage with a nucleotide sequence encoding a reporter, 2) an expression construct encoding a fusion protein, the fusion protein including a DNA binding domain and ELK1 lacking an ETS DNA binding domain wherein the DNA binding domain is substituted for the ETS DNA binding domain of ELK1; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the first recombinant cell and specifically binds to ELK1 in the DNA binding domain-ELK1 fusion protein, thereby activating expression of the reporter gene in the first recombinant cell; and II) a second recombinant cell, the second recombinant cell including: an androgen response element (ARE)-reporter gene expression construct including an ARE in operable linkage with a nucleic acid encoding a reporter, and an expression construct encoding human androgen receptor (AR), wherein in the presence of androgen, the AR enters the nucleus of the second recombinant cell and specifically binds to the ARE, thereby activating expression of the reporter gene in the second recombinant cell; contacting the first recombinant cell and the second recombinant cell with a test compound; and assaying the first recombinant cell and the second recombinant cell for expression of the reporter gene, wherein 1) partial or complete reversal of the activation of the reporter gene in the first recombinant cell in the presence of androgen and 2) no change in expression of the reporter gene in the second recombinant cell in the presence of androgen or a smaller reduction in reporter expression compared with the first recombinant cell, together indicate that the test compound inhibits interaction of the AR with ELK1, thereby identifying a compound characterized by anti-hormone receptor-dependent cancer activity. Optionally, the reporter gene expression construct; the expression construct encoding a fusion protein; the expression construct encoding AR, any two thereof, or all of these, is stably integrated in the genome of the first recombinant cell. Optionally, one or both of the ARE-reporter gene expression construct; and the expression construct encoding AR, is stably integrated in the genome of the second recombinant cell. Optionally, the reporter gene expression construct of the first recombinant cell and the reporter gene expression construct of the second recombinant cell both encode the same reporter. Optionally, the reporter gene expression construct of the first recombinant cell and the reporter gene expression construct of the second recombinant cell both encode firefly luciferase. Optionally, the reporter gene expression construct of the first recombinant cell and the reporter gene expression construct of the second recombinant cell encode different reporters. Optionally, the reporter gene expression construct of the first recombinant cell and the reporter gene expression construct of the second recombinant cell encode the same reporter and the first recombinant cell and the second recombinant cell are present in separate assay vessels or the reporter gene expression construct of the first recombinant cell and the reporter gene expression construct of the second recombinant cell encode different reporters and the first recombinant cell and the second recombinant cell are present in the same assay vessel.

An assay to identify a compound characterized by anti-hormone receptor-dependent cancer activity is provided according aspects of the present invention which include: I) a first recombinant cell having a nucleus, the first recombinant cell including: 1) a reporter gene expression construct including a DNA binding domain recognition sequence in operable linkage with a nucleotide sequence encoding a reporter, wherein the DNA binding domain recognition sequence includes a Gal4 DNA binding domain recognition sequence, or two or more contiguous Gal4 DNA binding domain recognition sequences; 2) an expression construct encoding a fusion protein, the fusion protein including a DNA binding domain and ELK1 lacking an ETS DNA binding domain, wherein the DNA binding domain is substituted for the ETS DNA binding domain of ELK1 and wherein the DNA binding domain of the fusion protein is a Gal4 DNA binding domain that specifically binds to the Gal4 DNA binding domain recognition sequence or to the two or more contiguous Gal4 DNA binding domain recognition sequences; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the first recombinant cell and specifically binds to ELK1 in the DNA binding domain-ELK1 fusion protein, thereby activating expression of the reporter gene in the first recombinant cell; and II) a second recombinant cell, the second recombinant cell including: an androgen response element (ARE)-reporter gene expression construct including an ARE in operable linkage with a nucleic acid encoding a reporter, and an expression construct encoding human androgen receptor (AR), wherein in the presence of androgen, the AR enters the nucleus of the second recombinant cell and specifically binds to the ARE, thereby activating expression of the reporter gene in the second recombinant cell. Optionally, the reporter gene expression construct; the expression construct encoding a fusion protein; the expression construct encoding AR, any two thereof, or all of these, is stably integrated in the genome of the first recombinant cell. Optionally, one or both of the ARE-reporter gene expression construct; and the expression construct encoding AR, is stably integrated in the genome of the second recombinant cell. Optionally, the reporter gene expression construct of the first recombinant cell and the reporter gene expression construct of the second recombinant cell both encode the same reporter. Optionally, the reporter gene expression construct of the first recombinant cell and the reporter gene expression construct of the second recombinant cell both encode firefly luciferase. Optionally, the reporter gene expression construct of the first recombinant cell and the reporter gene expression construct of the second recombinant cell encode different reporters. Optionally, the reporter gene expression construct of the first recombinant cell and the reporter gene expression construct of the second recombinant cell encode the same reporter and the first recombinant cell and the second recombinant cell are present in separate assay vessels or the reporter gene expression construct of the first recombinant cell and the reporter gene expression construct of the second recombinant cell encode different reporters and the first recombinant cell and the second recombinant cell are present in the same assay vessel.

A recombinant cell having a nucleus is provided according to aspects of the present invention which includes: 1) a reporter gene expression construct including a DNA binding domain recognition sequence in operable linkage with a nucleotide sequence encoding a reporter, 2) an expression construct encoding a fusion protein, the fusion protein including a DNA binding domain and ELK1 lacking an ETS DNA binding domain wherein the DNA binding domain is substituted for the ETS DNA binding domain of ELK1; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the first recombinant cell and specifically binds to ELK1 in the DNA binding domain-ELK1 fusion protein, thereby activating expression of the reporter gene in the first recombinant cell. Optionally, the reporter gene encodes firefly luciferase. Optionally, the reporter gene expression construct; the expression construct encoding a fusion protein; the expression construct encoding AR, two or more thereof, or all of these, is stably integrated in the genome of the recombinant cell.

A recombinant cell having a nucleus is provided according to aspects of the present invention which includes: 1) a reporter gene expression construct including a DNA binding domain recognition sequence in operable linkage with a nucleotide sequence encoding a reporter, wherein the DNA binding domain recognition sequence includes a Gal4 DNA binding domain recognition sequence, or two or more contiguous Gal4 DNA binding domain recognition sequences; 2) an expression construct encoding a fusion protein, the fusion protein including a DNA binding domain and ELK1 lacking an ETS DNA binding domain wherein the DNA binding domain is substituted for the ETS DNA binding domain of ELK1 and wherein the DNA binding domain of the fusion protein is a Gal4 DNA binding domain that specifically binds to the Gal4 DNA binding domain recognition sequence or to the two or more contiguous Gal4 DNA binding domain recognition sequences; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the first recombinant cell and specifically binds to ELK1 in the DNA binding domain-ELK1 fusion protein, thereby activating expression of the reporter gene in the first recombinant cell. Optionally, the reporter gene encodes firefly luciferase. Optionally, the reporter gene expression construct; the expression construct encoding a fusion protein; the expression construct encoding AR, two or more thereof, or all of these, is stably integrated in the genome of the recombinant cell.

Assay systems for identifying a compound characterized by anti-hormone receptor-dependent cancer activity are provided according to aspects of the present invention which include a recombinant cell having a nucleus, the recombinant cell including: 1) a first reporter gene expression construct including a DNA binding domain recognition sequence in operable linkage with a nucleotide sequence encoding a first reporter, 2) an expression construct encoding a fusion protein, the fusion protein including a DNA binding domain and ELK1 lacking an ETS DNA binding domain wherein the DNA binding domain is substituted for the ETS DNA binding domain of ELK1; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the recombinant cell and specifically binds to ELK1 in the DNA binding domain-ELK1 fusion protein, thereby activating expression of the first reporter gene in the recombinant cell; and the recombinant cell further including: an androgen response element (ARE)-reporter gene expression construct including an ARE in operable linkage with a nucleic acid encoding a second reporter; and an expression construct encoding human androgen receptor (AR), wherein in the presence of androgen, the AR enters the nucleus of the recombinant cell and specifically binds to the ARE, thereby activating expression of the second reporter gene in the recombinant cell, wherein the first and second reporter genes express detectably different reporters.

Assay systems for identifying a compound characterized by anti-hormone receptor-dependent cancer activity are provided according to aspects of the present invention which include a recombinant cell having a nucleus, the recombinant cell including: 1) a first reporter gene expression construct including a DNA binding domain recognition sequence in operable linkage with a nucleotide sequence encoding a first reporter, wherein the DNA binding domain recognition sequence includes a Gal4 DNA binding domain recognition sequence, or two or more contiguous Gal4 DNA binding domain recognition sequences; 2) an expression construct encoding a fusion protein, the fusion protein including a DNA binding domain and ELK1 lacking an ETS DNA binding domain wherein the DNA binding domain is substituted for the ETS DNA binding domain of ELK1 and wherein the DNA binding domain is a Gal4 DNA binding domain that specifically binds to the Gal4 DNA binding domain recognition sequence or to the two or more contiguous Gal4 DNA binding domain recognition sequences; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the recombinant cell and specifically binds to ELK1 in the DNA binding domain-ELK1 fusion protein, thereby activating expression of the first reporter gene in the recombinant cell; and the recombinant cell further including: an androgen response element (ARE)-reporter gene expression construct including an ARE in operable linkage with a nucleic acid encoding a second reporter, and an expression construct encoding human androgen receptor (AR), wherein in the presence of androgen, the AR enters the nucleus of the recombinant cell and specifically binds to the ARE, thereby activating expression of the second reporter gene in the recombinant cell, wherein the first and second reporter genes express detectably different reporters. Optionally, one or more of: the first reporter gene expression construct; the expression construct encoding a fusion protein; the expression construct encoding AR, the ARE-reporter gene expression construct; and the expression construct encoding AR, or any two or more thereof, or all of these, is stably integrated in the genome of the recombinant cell.

### BRIEF DESCRIPTION OF THE DRAWINGS

FIG. 1A shows a schematic of a reporter system for the primary screening assay;

FIG. 1B shows a schematic of the reporter system for the counter screening assay;

FIG. 2A shows results from a primary screen in which recombinant HeLa cells described for FIG. 1A were treated; and

FIG. 2B shows results from a counter screen in which recombinant HeLa cells described for FIG. 1B were treated.

### DETAILED DESCRIPTION OF THE INVENTION

ELK1 is a steroid hormone receptor tethering protein that is implicated in hormone receptor dependent cancers. Assays are provided according to aspects of the present invention which identify inhibitors of the association of steroid hormone receptors with ELK1, inhibiting steroid-receptor dependent gene activation and providing therapeutic activity against steroid hormone-receptor-dependent cancer.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, P A, 2003; Herdewijn, P. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, 3rd edition, Cold Spring Harbor Laboratory Press; Dec. 15, 2002, ISBN-10: 0879695919; Kursad Turksen (Ed.), Embryonic stem cells: methods and protocols in Methods Mol Biol. 2002; 185, Humana Press; Current Protocols in Stem Cell Biology, ISBN: 9780470151808; Chu, E. and Devita, V. T., Eds., Physicians' Cancer Chemotherapy Drug Manual, Jones & Bartlett Publishers, 2005; J. M. Kirkwood et al., Eds., Current Cancer Therapeutics, 4th Ed., Current Medicine Group, 2001; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, Pa.: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

The term “nucleic acid” refers to RNA or DNA molecules having more than one nucleotide in any form including single-stranded, double-stranded, oligonucleotide, or polynucleotide. The term “nucleotide sequence” refers to the ordering of nucleotides in a nucleic acid.

The term “hormone-sensitive cancer” as used herein refers to a cancer that is dependent on a steroid hormone for growth and/or survival. The term “hormone-receptor-dependent cancer” as used herein includes steroid hormone-sensitive cancer as well as cancers that depend on a steroid receptor for a steroid hormone acting independent of the steroid hormone or that depend on variant forms of the steroid hormone receptor that cannot bind steroid hormone. The terms “hormone-sensitive cancer” and “hormone-receptor-dependent cancer” include, but are not limited to, estrogen-sensitive cancers or otherwise estrogen receptor-dependent cancers and androgen-sensitive cancers or otherwise androgen receptor-dependent cancers. Non-limiting examples of hormone sensitive cancers and otherwise hormone-receptor-dependent cancers include testosterone-sensitive prostate cancer and testosterone-independent but androgen receptor-dependent prostate cancer including castration resistant prostate cancer (CRPC). Further examples include estrogen-sensitive breast cancer and estrogen-independent but estrogen receptor-dependent breast cancer. Assay systems and methods are provided according to aspects of the present invention which identify inhibitors of the association of steroid hormone receptors with ELK1, inhibiting steroid-receptor dependent gene activation and providing therapeutic activity against steroid hormone-receptor-dependent cancer.

Assay Systems and Methods

Assay systems for identifying a compound characterized by anti-hormone receptor-dependent cancer activity are provided according to aspects of the present invention which include: A) a first recombinant cell having a nucleus, the first recombinant cell comprising: 1) a reporter gene expression construct including a DNA binding domain recognition sequence in operable linkage with a nucleotide sequence encoding a reporter; 2) an expression construct encoding a fusion protein, the fusion protein including a DNA binding domain and ELK1 lacking an ETS DNA binding domain wherein the DNA binding domain is substituted for the ETS DNA binding domain of ELK1; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the first recombinant cell and specifically binds to ELK1 in the DNA binding domain-ELK1 fusion protein, thereby activating expression of the reporter gene in the first recombinant cell; and B) a second recombinant cell, the second recombinant cell including: an androgen response element (ARE)-reporter gene expression construct including an ARE in operable linkage with a nucleic acid encoding a reporter, and an expression construct encoding human androgen receptor (AR), wherein in the presence of androgen, the AR enters the nucleus of the second recombinant cell and specifically binds to the ARE, thereby activating expression of the reporter gene in the second recombinant cell.

Optionally, reporter gene expression constructs are used which produce detectably different reporters such that all of A and B are present in the same recombinant cell.

According to particular aspects, the DNA binding domain recognition sequence of the reporter gene expression construct of the first recombinant cell includes one or more contiguous Gal4 DNA binding domain recognition sequences; wherein the DNA binding domain of the fusion protein is a Gal4 DNA binding domain that specifically binds to the one or more Gal4 DNA binding domain recognition sequences.

According to particular aspects one, two or all of the expression constructs selected from the group consisting of: the reporter gene expression construct; the expression construct encoding a fusion protein; the expression construct encoding AR, is stably integrated in the genome of the first recombinant cell.

According to particular aspects one or both of the expression constructs selected from the group consisting of: the ARE-reporter gene expression construct; and the expression construct encoding AR, is stably integrated in the genome of the second recombinant cell.

Optionally, the reporter gene expression construct of the first recombinant cell and the reporter gene expression construct of the second recombinant cell both encode the same reporter. According to particular aspects, the reporter gene expression construct of the first recombinant cell and the reporter gene expression construct of the second recombinant cell both encode firefly luciferase. Accordingly, assay of reporter expression of the first recombinant cell and the second recombinant cell are performed separately, such as in separate assay vessels, to distinguish the expression of their respective reporters.

Optionally, the reporter gene expression construct of the first recombinant cell and the reporter gene expression construct of the second recombinant cell encode different reporters. Accordingly, assay of reporter expression of the first recombinant cell and the second recombinant cell are performed together or separately, such as in the same or separate assay vessels, to distinguish the expression of their respective reporters.

Assay vessels for performing an assay include, but are not limited to, cell culture plates.

Recombinant cells described herein in detail are genetically modified HeLa cells. Other suitable cell types which can be used include mammalian cells which are “steroid hormone receptor positive.” The term “steroid hormone receptor positive” refers to cells which express a particular steroid hormone receptor of interest, such as estrogen receptor and/or androgen receptor. The cells natively express steroid hormone receptor or are genetically modified to express the steroid hormone receptor of interest. As noted herein, the hormone receptor of interest is stably integrated via an expression construct into the cells according to aspects of the present invention.

Regarding the reporter gene nucleic acid expression construct and nucleic acid expression construct encoding a DNA binding domain-ELK1 fusion protein wherein the ETS DNA binding domain of ELK1 is not present and an exogenous DNA binding domain is substituted for the ETS DNA binding domain of ELK1 of the first recombinant cell, the exogenous DNA binding domain specifically binds to the DNA binding domain recognition sequence of the reporter gene expression construct. The identity of the DNA binding domain recognition sequence and exogenous DNA binding domain is not limited and can be any suitable DNA binding domain recognition sequence/DNA binding domain pair which specifically bind to each other. According to aspects described herein, the DNA binding domain recognition sequence is a Gal4 DNA binding domain recognition sequence and the DNA binding domain is a Gal4 DNA binding domain, both of which are well-known in the art, along with variants of each which can be used. More than one Gal4 DNA binding domain recognition sequence is optionally included, such as 2, 3, 4, 5 or more contiguous Gal4 DNA binding domain recognition sequences.

Nucleotide sequences encoding human androgen receptor (AR) and androgen response element (ARE) are well-known in the art, along with variants of each which can be used. In particular, the amino-terminal A/B domain of AR [AR(A/B)] is sufficient to bind to ELK1, such that AR splice variants containing the AR(A/B) or the AR(A/B) alone are optionally encoded in a nucleic acid expression construct stably incorporated in the genome of the first recombinant cell according to aspects of the present invention.

ELK1 is a downstream effector of the MAPK signaling pathway and belongs to the ternary complex factor (TCF) sub-family of the ETS family of transcription factors. ELK1 characteristically binds to purine-rich GGA core sequences, see Shaw P E, et al., Int. J. Biochem. Cell Biol., 2003; 35(8):1210-26. ELK1 is in a repressive association with many cell growth genes. Phosphorylation by ERK transiently stimulates ELK1 to activate its target genes including association with serum response factor (SRF) for activation of immediate early genes, see Shaw P E, et al., Int. J. Biochem. Cell Biol., 2003; 35(8):1210-26; Sharrocks A D., Nature Rev. Mol. Cell Biol., 2001; 2(11):827-37; Shaw P E, et al., EMBO J., 1989; 8(9):2567-74; Gille H, et al., Nature, 1992; 358(6385):414-7; Gille H, et al., EMBO J., 1995; 14(5):951-62; and Zhang H M, et al., NAR, 2008; 36(8):2594-607. The N-terminal A/B domain of AR [AR(A/B)], which lacks the ligand binding site, is adequate for interaction with ELK1, see Patki M, et al., J. Biol. Chem., 2013; 288(16):11047-65. AR splice variants, which have C-terminal deletions and lack the ligand binding domain (LBD), also synergize with ELK1 and support growth, Rosati R, et al., J. Biol. Chem., 2016; 291 (50):25983-25998.

Nucleotide sequences encoding human ELK1 are well-known in the art, along with variants which can be used. Systematic in situ mapping of the ELK1 polypeptide using mammalian two-hybrid assays precisely identified its two ERK docking sites [D-box and DEF (Docking site for ERK, FXFP) motif], and excluded its transactivation domain, as the essential motifs for its cooperation with AR(A/B), wtAR and AR-V7. Surface plasmon resonance (SPR) showed direct binding of purified ELK1 and AR with a dissociation constant of 1.9×10⁻⁸ M. A purified mutant ELK1 in which the D-box and DEF motifs were disrupted did not bind AR. An ELK1 mutant with deletion of the D-box region had a dominant-negative effect on androgen-dependent growth of PCa cells that were insensitive to MEK inhibition.

Compositions and methods of the present invention are not limited to particular amino acid and nucleic sequences identified by SEQ ID NO herein and variants of a reference nucleic acid or protein may be used.

As used herein, the term “variant” refers to naturally occurring genetic variations and recombinantly engineered variations in a nucleotide sequence or amino acid sequence which contains one or more changes in its sequence compared to a specified reference sequence while retaining the desired functional properties of the specified reference sequence.

Such changes include those in which one or more amino acid residues have been modified by amino acid substitution, addition or deletion. The term “variant” encompasses orthologs of human nucleotide sequences and amino acid sequences described herein, including for example those derived from mammals and birds, such as, but not limited to orthologs from a non-human primate, cat, dog, sheep, goat, horse, cow, pig, bird, poultry and rodent such as but not limited to mouse and rat.

Mutations can be introduced using standard molecular biology techniques, such as site-directed mutagenesis and PCR-mediated mutagenesis. One of skill in the art will recognize that one or more nucleotide sequence mutations or amino acid sequence mutations can be introduced without altering the functional properties of the specified reference nucleotide sequence or specified reference protein.

Variants of a specified nucleic acid encoding a specified protein described herein are nucleic acids having a nucleotide sequence that has at least 70%, 75%, 80%, 85%, 90%/, 91%, 92%, 93%, 94%, 95%, 96%, 97%, 98%, 99% or greater, identity to a nucleotide sequence set forth herein. Variants of a specified nucleotide sequence can encode an amino acid sequence identical to that of a specified protein (due to degeneracy of the genetic code) or a variant of the specified protein.

Variants of a specified protein described herein are proteins having an amino acid sequence that has at least 70%, 75%, 80%, 85%, 90%, 91%, 92%, 93%, 94%, 95%, 96%, 97%, 98%, 99% or greater, identity to an amino acid sequence set forth herein.

Conservative amino acid substitutions can be made in a specified protein to produce a protein variant. Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

Protein variants can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-bydroxytryptophan, 1-methyihistidine, 3-methylhistidine, and ornithine.

To determine the percent identity of two amino acid sequences or of two nucleotide sequences, the sequences are aligned for optimal comparison purposes (e.g., gaps can be introduced in the sequence of a first amino acid or nucleotide sequence for optimal alignment with a second amino acid or nucleotide sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, PNAS 87:2264 2268, modified as in Karlin and Altschul, 1993, PNAS. 90:5873 5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, wordlength=12 to obtain nucleotide sequences homologous to a nucleic acid molecules of the present invention. BLAST protein searches are performed with the XBLAST program parameters set, e.g., to score 50, wordlength=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389 3402. Alternatively, PSI BLAST is used to perform an iterated search which detects distant relationships between molecules (Id.). When utilizing BLAST, Gapped BLAST, and PSI Blast programs, the default parameters of the respective programs (e.g., of XBLAST and NBLAST) are used (see, e.g., the NCBI website). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

Proteins and nucleic acids described herein may be generated recombinantly, such as by expression using an expression construct. Proteins and nucleic acids may also be chemically synthesized by well-known methods.

A reporter encoded by expression constructs in the first and second recombinant cells can be any of various reporters that can be used to produce a detectable signal indicative of expression, such as, chloramphenicol acetyltransferase (CAT), alkaline phosphatase, beta-galactosidase, luciferase, and fluorescent proteins such as, Green Fluorescent Protein (GFP), yellow fluorescent protein, cyan fluorescent protein, DsRed and fluorescent variants of any thereof. Methods of detecting a detectable signal of a reporter are well-known in the art.

An expression construct is introduced into the genome of a cell producing a stably integrated genetic modification of the genome of the cell according to aspects of the present invention. The term “stable,” “stably integrated” and grammatical equivalents in reference to genetically modified cells refers to the long-term or permanent integration of exogenous DNA into the genome of the cell. Insertion of an expression construct in a cell genome can be confirmed by various well-known methods, including PCR and Southern blot analysis.

The term “genetically modified” and grammatical equivalents in reference to genetically modified cells as used herein refers to the introduction of an exogenous nucleic acid into a cell by genetic engineering techniques or a descendant of such a cell that has inherited at least a portion of the introduced exogenous nucleic acid.

An expression construct is introduced into target cells in order to produce a cell including the expression construct in the genome of the cell using well-known methods, such as lentivirus transduction or other viral transduction methods, microinjection, electroporation, calcium-phosphate precipitation, or lipofection.

Following transduction or transfection, the cells are grown in a medium optimized for the particular cell line. Transduced or transfected cells are typically selected for by including an antibiotic in the medium according to well-established methods.

Integration of an expression construct into the genome of a cell can be determined by genetic analysis, such as PCR, Southern blot, or nucleotide sequencing, and expression of an encoded protein can be determined by protein expression analysis such as by protein analysis (immunocytochemistry, Western blot, ELISA) and/or functional assays.

The terms “expression construct” and “expression cassette” are used herein to refer to a double-stranded recombinant nucleotide sequence containing a desired coding sequence and containing one or more regulatory elements necessary or desirable for the expression of the operably-linked coding sequence. The term “regulatory element” as used herein refers to a nucleotide sequence that controls some aspect of the expression of nucleotide sequences. Exemplary regulatory elements illustratively include an enhancer, a TATA box, an internal ribosome entry site (IRES), an intron, an origin of replication, a polyadenylation signal (pA), a promoter, a transcription termination sequence, and an upstream regulatory domain, which contribute to the replication, transcription, and post-transcriptional processing of a nucleotide sequence. Those of ordinary skill in the art are capable of selecting and using these and other regulatory elements in an expression construct with no more than routine experimentation. Expression constructs may be generated recombinantly or synthetically using well-known methodology.

The term “operably-linked” as used herein refers to a nucleotide sequence in a functional relationship with a second nucleotide sequence.

A regulatory element included in an expression cassette may be a promoter. The term “promoter” as used herein refers to a regulatory nucleotide sequence operably-linked to a coding nucleotide sequence to be transcribed such as a nucleotide sequence encoding a desired sequence of amino acids. A promoter is generally positioned upstream of a nucleotide sequence to be transcribed and provides a site for specific-binding by RNA polymerase and other transcription factors. A promoter may be a constitutive promoter or an inducible promoter. A promoter may provide ubiquitous, tissue-specific, or cell-type specific expression.

In addition to a promoter, one or more enhancer sequences may be included such as, but not limited to, the cytomegalovirus (CMV) early enhancer element and the SV40 enhancer element.

Additional included sequences include an intron sequence such as the beta globin intron or a generic intron, a transcription termination sequence, and an mRNA polyadenylation (pA) sequence such as, but not limited to SV40-pA, beta-globin-pA, and AAT-pA.

An expression construct may include sequences necessary for amplification in bacterial cells, such as a selection marker (e.g., a kanamycin or ampicillin resistance gene) and an origin of replication.

Assays to identify a compound characterized by anti-hormone receptor-dependent cancer activity, including: providing the first recombinant cell and the second recombinant cell; contacting the first recombinant cell and the second recombinant cell with a test compound; and determining the effect of the test compound on expression of a reporter gene in the first recombinant cell and the second recombinant cell compared to an appropriate control, wherein 1) a decrease in expression of the reporter gene in the first recombinant cell compared to a positive control and 2) no change in expression of the reporter gene in the second recombinant cell compared to the positive control, together indicate that the test compound inhibits interaction of the AR with ELK1, thereby identifying a compound characterized by anti-hormone receptor-dependent cancer activity.

Controls are well-known in the art and one of skill in the art would readily recognize an appropriate control and be able to determine an appropriate control for a method of the present invention with no more than routine experimentation.

Testosterone can be used as a negative control in an assay using an assay system according to aspects of the present invention. In the first recombinant cell of the primary screen, testosterone interacts with the AR causing the AR to enter the nucleus. In the nucleus, the AR binds to the DNA binding domain-ELK1 fusion protein, which is bound to the DNA binding domain recognition sequence of the reporter gene expression construct, thereby activating expression of the reporter gene in the first recombinant cell. Testosterone is likewise a negative control in the second recombinant cell in which testosterone interacts with the AR causing the AR to enter the nucleus. In the nucleus, the AR binds to the androgen response element of the androgen response element-reporter gene expression construct, thereby activating expression of the reporter gene in the second recombinant cell.

A positive control can be used. A positive control in this context is a compound that completely inhibits the effect of testosterone in both the primary and counter screen assays. An example of a useful positive control compound in these assays is enzalutamide, which competes with testosterone and binds to AR preventing AR from entering the nucleus. Enzalutamide is regarded as the positive control as it produces the effect that an inhibitory compound would.

An appropriate control may be a reference level of reporter gene expression obtained from similar tests previously and stored in a print or electronic medium for recall and comparison.

A reporter gene included in the first recombinant cell and the second recombinant cell can be the same or different, producing the same or different detectable signal. Where the reporter gene included in the first recombinant cell and the second recombinant cell is different, the cells are optionally assayed for response to a test substance in the same assay vessel and the different detectable signals from different reporter genes in the first and second recombinant cells are detected. Where the reporter gene included in the first recombinant cell and the second recombinant cell is the same, the cells are assayed for response to a test substance in different assay vessels and the detectable signals from the reporter genes in the first and second recombinant cells are detected.

A test compound used in a method of the present invention can be any chemical entity, illustratively including a synthetic or naturally occurring compound or a combination of a synthetic or naturally occurring compound, a small organic or inorganic molecule, a protein, a peptide, a nucleic acid, a carbohydrate, an oligosaccharide, a lipid or a combination of any of these. A test compound may be a naturally-occurring or synthetic chemical compound, or a mixture of two or more thereof. A test compound can be an extract of an environmental sample or organism, such as soil, bacteria, insects or plants, which contain several characterized or uncharacterized components. The components can be further isolated and tested to identify one or more of the components with the desired activity.

In one embodiment, the test compound is a small molecule compound. Individual small molecule test compounds may be identified from combinatorial chemistry libraries, and then further optimized through chemical alterations if desired.

The amount of test compound used in an assay according to aspects of the present invention is generally in the range of about 0.01 nM to 1 mM, for example from 0.1 nM to 100 μM, but more, or less, can be used.

Embodiments of inventive compositions and methods are illustrated in the following examples. These examples are provided for illustrative purposes and are not considered limitations on the scope of inventive compositions and methods.

### Examples

Cell Culture and Reagents

HeLa cell lines were obtained from the American Type Culture Collection (Manassas, Va.). HeLa cells were grown in DMEM medium supplemented with 10% FBS (Invitrogen), 100 units/ml penicillin, 100 μg/ml streptomycin, 2 mM L-glutamine mixture (Invitrogen). Testosterone was obtained from Sigma-Aldrich. Lipofectamine™ 2000 was purchased from Thermo Scientific (product number 78410).

Gal4 DNA binding domain recognition sequence, vector containing luciferase reporter gene attached to a TATA box containing promoter and the manner in which the Gal4-TATA-Luciferase construct was generated

Custom synthesized PCR primers were used to amplify and clone the five tandem Gal4 elements from the pG5luc vector (Promega) into the pGreenFire1TM-mCMV-EF1-Neo (Plasmid) at SpeI (upstream) and BamHI (downstream) sites. pG5luc Vector is well known and the complete sequence has NCBI accession #AF264724

Generation of the Gal4-ELK1 Fusion Protein Construct

The Gal4 fusion with ELK1 in which the DNA binding domain of ELK1 (amino acids 1-86) was deleted was constructed by PCR using the ELK-pCMV expression plasmid (Origene, Rockville, Md.) as the template and the appropriate primers subcloned at BamHI (upstream) and NotI (downstream) sites in a vector expressing Gal4 fusions (pBind). The Gal4ELK1 fusion sequence was then cloned into the pCDH-CMV-MSC-EF1-Hygro cDNA cloning and expression vector at XbaI site (upstream) and BamHI (downstream) sites.

Generation of Recombinant Cell Lines for High Throughput Screening and Counter Screening of Small Molecule Libraries

HeLa HLR cells obtained from Dr. Johann Hofman (Innsbruck Medical University), which were originally designed to serve as a cell-based assay system to measure modulation of MAPK activity. HeLa HLR cells have a stably integrated minimal promoter-luciferase reporter containing five upstream Gal4 elements (Gal4-TATA-Luc) and also constitutively express a Gal4-ELK1 fusion protein in which the Gal4 DNA binding domain is substituted for the ETS DNA binding domain of ELK1. In order to produce recombinant cell lines for high throughput screening and counter screening of small molecule libraries, HeLa HLR were stably transduced with a vector expressing the full-length AR. A sequence encoding the full length AR (SEQ ID NO:9) was subcloned from the pCMV expression vector (Origene) into the pCDH-CMV-MCS-EF1-Puro cDNA Cloning and Expression Vector (System Biosciences) at NheI (upstream) and BamHI (downstream) sites. The lentiviral vector expressing full length AR was then packaged in lentivirus and the HeLa HLR cells were infected as described below in the sub-section “Lentivirus-mediated-Transduction.” After 72 hours (01) of infection, 2 ug/mL of puromycin was added to the culture medium to select for the transduced cells. The cells were plated at low density for colony formation (20-40 colonies) in a 100 mm dish. Clonal cells were isolated using cloning cylinders from CORNING (Cat. #3166-8). The selected clones were further expanded and then tested for luciferase induction by testosterone. The clone that gave the greatest luciferase signal to noise ratio in response to testosterone treatment was then chosen for use in the primary screening assay for high throughput small molecule screening.

The cells generated for counter screening are HeLa cells stably transduced with a lentiviral plasmid construct containing a minimal promoter-luciferase reporter and an upstream androgen response element (ARE) sequence. These HeLa cells were also transduced with a lentiviral expression plasmid encoding the full-length AR. These lentiviral constructs were made as follows. Custom synthesized PCR primers were used to amplify and clone the ARE sequence (SEQ ID NO:11) from a pG5luc plasmid construct containing an ARE sequence element into the pGreenFire1TM-mCMV-EF1-Neo (Plasmid) at SpeI (upstream) and BamHI (downstream) sites. pG5luc Vector is well known and the complete sequence has NCBI accession #AF264724. The ARE containing pG5luc plasmid is described in detail in Patki et al., 2013, J Biol Chem, 288:11047-11065. The lentiviral vector expressing ARE-luciferase reporter was then packaged in lentivirus and parental HeLa cells were infected as described below under the heading “Lentivirus-mediated-Transduction.” After 72 h of infection, 400 ug/mL of Geneticin was added to the culture medium to select for the transduced cells. These cells were then infected with the lentivirus containing the full length AR expression plasmid described above. After 72 h of infection, 2 ug/mL of Puromycin was added to the culture medium to select for the transduced cells. Clonal cells harboring both the ARE-promoter-luciferase reporter and also stably expressing AR were then isolated using cloning cylinders as described above. The selected clones were further expanded and then tested for luciferase induction by testosterone. The clone that gave the greatest luciferase signal to noise ratio in response to testosterone treatment was then chosen for use in the counter screening assay for high throughput small molecule screening. All of the plasmid constructs generated above were sequenced to verify DNA sequences before the constructs were used.

The recombinant HeLa cells generated above were routinely grown in DMEM supplemented with 10% FBS and 100 units/ml penicillin, 100gig/ml streptomycin, 2 mM L-glutamine mixture (Invitrogen) and the appropriate selection antibiotics. The antibiotics used in the culture media for the primary screening cells included 100 μg/ml Hygromycin (Invitrogen) (to maintain Gal4-ELK1), 100 μg/ml Geneticin (Invitrogen) (to maintain Gal4-TATA-Luc) and 2 μg/ml Puromycin (Sigma-Aldrich) (to maintain AR). The antibiotics used in the culture media for the counter screening cells included 400 ug/ml Geneticin (Invitrogen) (to maintain ARE-TATA-Luc) 2 μg/ml Puromycin (Sigma-Aldrich) (to maintain AR).

High Throughput Screening

For high throughput screening, recombinant primary screening cells were first depleted of hormone by growing them for 24 h in medium in which the serum used was heat-inactivated and charcoal-stripped. The cells were then plated in 384-well white flat bottom plates (5,000 cells/well) (Corning Product #3570) using a Multidrop (Thermo Fisher Scientific, Waltham, Mass.). The plates were then incubated for 24 h prior to adding test compounds. The following day test compounds from the LOPAC, Prestwick, or Maybridge Hitfinder libraries were added precisely in a 0.2 μL volume in the test wells using a Biomek FX liquid handler (Beckman Coutler, Break, Calif.) to achieve final media concentration of 10 μM of each test compound. Using the same technique, testosterone was added in addition to the test compounds to achieve a final media concentration of 10 nM. As the test compounds were re-constituted from powder stocks using dimethyl sulfoxide (DMSO) as the solvent, the final media concentration of DMSO was 0.4% v/v. For the assay negative control on each plate, one row of wells on each plate contained 10 nM testosterone and 0.4% v/v of DMSO. For the assay positive control on each plate, one row of wells on each plate contained 10 nM testosterone and 10 uM enzalutamide dissolved in DMSO (0.4% v/v of DMSO in the wells). The plates were incubated for 24 h at 37° C. in 5% CO₂. The medium was then aspirated leaving a residual volume 10 μl using an Elx 405-plate washer (Bio Tek U.S.). Then, 10 uL of the assay reagent Bright-Glo (Promega Corp., Madison Wis.) was added to each well. Luciferase activities in the wells were then measured using a Biomek FX dual head (Beckman) plate reader). A total of 18,270 compounds were tested in the primary screen. A ‘hit’ compound was initially defined using relatively low stringency criteria as a compound able to reduce luciferase activity in the test well ≥3 standard deviations below the negative control wells or to a level ≥40% of the enzalutamide control wells. For the primary assay this definition produced 1613 hits for an overall hit rate of 8.8%. The 1613 compounds were then tested again in the primary screening assay in parallel with the counter screening assay in triplicate. A hit was now redefined as a test compound able to reduce luciferase activity in the test wells ≥3 standard deviations below the negative control wells and that was unable to reduce luciferase activity ≥50% in the counter screen. By this definition, 92 hits were obtained. Compounds were further triaged based on their ability to reduce luciferase activity in the primary screen by ≥80% and produced no inhibition in the counter screen.

Transfections and Reporter Luciferase Assays

Hela Cells were plated in a 24-well plate at a concentration of 75,000 cells/well in antibiotic-free red DMEM. The following day the cells were transfected with a total of 300 ng of plasmid DNA/well using Lipofectamine 2000. The cells were incubated for 24 h then lysed using luciferase assay lysis buffer 5× from Promega. The luciferase activity of the cell lysates were measured using firefly substrate from Promega and a luminometer (Lumat LB9501, Berthold, Wildbad, Germany).

Lentivirus-Mediated-Transduction

The lentiviral vector expressing ARE-luciferase reporter was packaged in 293FT cells. The lentiviral particles were generated using lipofectamine and three plasmids, pMD2G, pMDLg/RRE, and pRSV/Rev which all code for essential elements of the virus. The virus containing supernatant was harvested at 48 and 72 h after transfection. Cells were plated in 6-well poly-D-lysine coated plates (BD Falcon) in phenol red-free medium supplemented with 10% heat-inactivated charcoal-stripped FBS and 2 mM L-glutamine. The following day, cells were infected with ARE-luciferase reporter lentivirus with Polybrene (8 μg/ml) for a 5 h duration, followed by an additional 5 h. After infection, the virus was replaced with fresh phenol red-free growth medium.

Colony Growth Assay

Cells were trypsinized, and 1000 cells/well were seeded in poly-D-lysine coated 6-well plates in phenol red-free regular growth media. The cells were treated with various concentrations of a “hit” compound, which was replenished every 48 h. The cells were grown at 37° C. in 5% CO2 for 10 days until colonies grew to the desired size in the untreated control wells. Colonies were fixed with methanol and stained with crystal violet. Each treatment was conducted in triplicate and the number of colonies was counted using the GelCount™ colony counter and a 350 size cutoff. Cell Monolayer Growth Assay.

Cells were trypsinized and 3000-4000 cells/well were seeded in 96-well plates coated with poly-D-lysine. The cells were seeded in phenol red-free medium supplemented with 10% FBS, 100 units/ml penicillin, 100 μg/ml streptomycin, 2 mM L-glutamine mixture and sodium pyruvate (1 mM) for LNCaP cells and phenol red-free medium supplemented with 10% FBS, 100units/ml penicillin, 100 μg/ml streptomycin and 2 mM L-glutamine mixture for VCaP, 22Rv1, DU145, HeLa, HEK293 and H1650 cells. The cells were grown at 37° C. in 5% CO₂. Twenty four hours after seeding in the 96-well plates, the cells were treated with various concentrations of a “hit” compound or DMSO (vehicle). The cells were re-treated on Day 3 by removing half the volume of medium and replacing it with fresh treated medium. Cell viability was determined using the MTT assay from day zero until day five. MTT (10 μL, 5 mg/mL) was added to each well and incubated for 2 h at 37° C. The formazan crystal sediments were dissolved in 100 μL of DMSO, and the absorbance at 570 nm was measured using the BioTek Synergy 2 Microplate Reader (BioTek, Winooski, Vt.). The assay was conducted in sextuplicate wells and values were normalized to day zero.

Tumor Xenograft Model Studies

The 22Rv1 human CRPC xenograft model was established by subcutaneous (SC) implant of 22Rv1 cells and serial passaging of the tumors in male SCID mice. A “hit” compound was administered by intraperitoneal (ip) injection (formulation: 1% carboxymethyl cellulose, 5% DMSO, and 0.5% NaHCO₃). For preliminary dose determinations, mice were tested for immediate post-injection toxicity by monitoring weight and behavior following daily ip injection of 3 doses of the compound (100, 150 or 250 mg/Kg body weight) for a duration of 7 days. As the mice were asymptomatic at all doses, the highest dose of 250 mg/Kg body weight was used for anti-tumor efficacy studies of the “hit” compound. Plasma levels of unmetabolized compound for this dose regimen was determined by LCMS as described in a separate sub-section. Male SCID mice were implanted bilaterally SC with 30-50 mg tumor fragments by 12 gauge trocar, and randomly distributed to various treatment and vehicle control groups. Treatment typically began 5 days post-implant (early stage disease) to determine antitumor efficacies and to further evaluate potential cumulative toxicities. Tumors were measured with a caliper 3 times/week and tumor masses (in mg) estimated by the formula, mg=(a×b2)/2, where “a” and “b” are tumor length and width in mm, respectively. Mice were sacrificed when cumulative tumor burdens reached 5-10% of body weight (1-2 g).

Results

Screening of Test Compounds

For the primary screen, recombinant AR+ HeLa cells harboring a reporter gene expression construct including 5 contiguous Gal4 DNA binding domain recognition sequences in operable linkage with a nucleic acid sequence encoding a reporter, 2) an expression construct encoding a fusion protein, the fusion protein including a Ga4 DNA binding domain and ELK1 lacking an ETS DNA binding domain wherein the Gal4 DNA binding domain is substituted for the ETS DNA binding domain of ELK1; and 3) an expression construct encoding human androgen receptor (AR); wherein the DNA binding domain of the DNA binding domain-ELK1 fusion protein specifically binds to the DNA binding domain recognition sequence of the reporter gene expression construct were used. When these cells are treated with testosterone, AR translocates to the nucleus where it binds to the Gal4-ELK1 fusion protein and activates the reporter gene. The cells for counter screening were identical to the primary screening cells with the exception that an androgen response element (ARE) sequence replaced the 5 contiguous Gal4 DNA binding domain recognition sequences in the promoter of the reporter gene and Gal4-ELK1 was absent. Compounds of interest should only suppress the signal in the primary screening assay, as the only difference between these two assays is AR recruitment to the promoter via ELK1 binding vs. direct DNA binding.

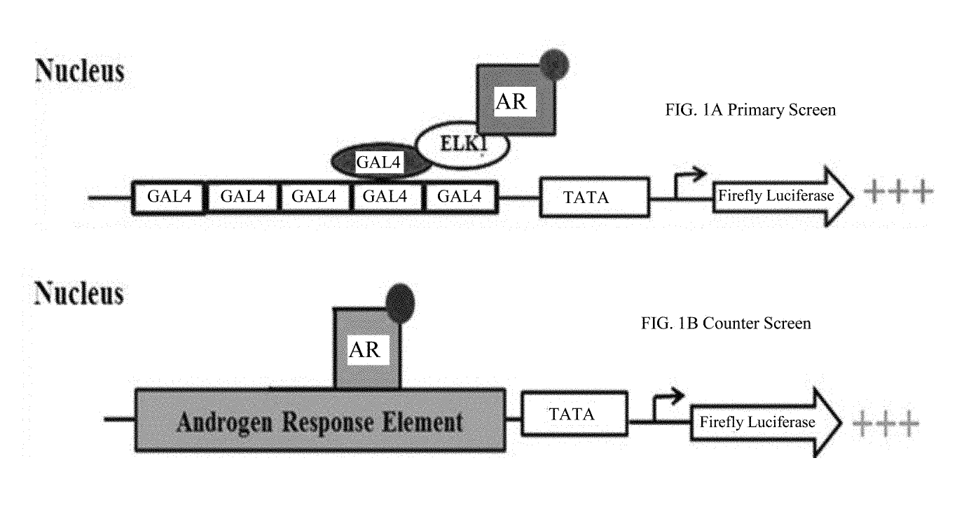

FIG. 1A shows a schematic of a reporter system for the primary screening assay. Recombinant HeLa cells used in this assay harbor the Gal4-TATA-luc promoter-reporter, and express a Gal4-ELK1 fusion protein as well as the androgen receptor (AR). Gal4-ELK1 is bound to the Gal4 elements in the promoter. In the absence of testosterone (small circle), AR is localized in the cytoplasm. When testosterone is present it binds to AR causing AR to translocate to the nucleus where it then binds to Gal4-ELK1 and activates the downstream luciferase reporter.

FIG. 1B shows a schematic of the reporter system for the counter screening assay. Recombinant HeLa cells used in this assay are identical to the primary screening cells except for the absence of Gal4-ELK1 and substitution of the Gal4 elements in the promoter with a canonical ARE. In this case, testosterone causes cytosolic AR to translocate to the nucleus and bind as a dimer to the ARE in the promoter resulting in activation of the luciferase reporter.

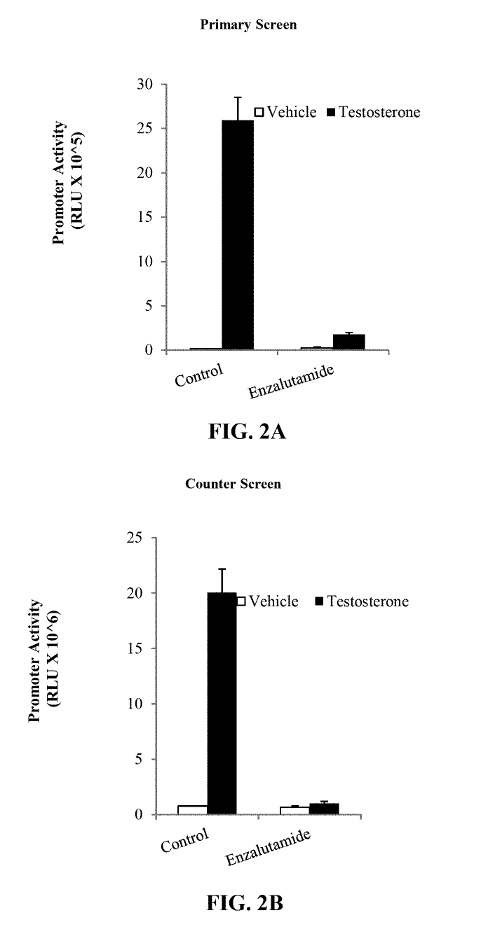

The Z-factor for the primary screening assay was 0.734 and for the counter screening assay it was 0.711. In both assays enzalutamide, which does not allow nuclear translocation of AR, completely suppressed the signal, see FIGS. 2A and 2B.

FIG. 2A shows results from a primary screen in which recombinant HeLa cells described for FIG. 1A were treated with either vehicle (ethanol) or 10 nM testosterone together with 10 uM of Enzalutamide or vehicle (DMSO) control. Cells were harvested and luciferase assay was performed 24 h post-treatment.

FIG. 2B shows results from a counter screen in which recombinant HeLa cells described for FIG. 1B were treated with either vehicle (ethanol) or 10 nM testosterone together with 10 uM of Enzalutamide or vehicle (DMSO) control. Cells were harvested and luciferase assay was performed 24 h post-treatment.

Two pilot sets of compound libraries, LOPAC and Prestwick, and then the Maybridge Hit Finder library, all of which are diversity sets, were screened at a compound concentration of 10 μM. A hit in the primary screen was defined as a compound able to reduce luciferase reporter activity ≥3 standard deviations below the negative control or to a level ≥40% of the enzalutamide control. This definition produced 1613 hits. Elimination of false positives by counter-screening resulted in 15 compounds with variable potencies (40%-100% inhibition) in the primary screen. Compounds were then selected for further testing based on potency of inhibition in the primary screen (80%-100% in <6 hours) and virtual absence of an effect in the counter screen. Compounds identified by the assay were confirmed to have specific inhibitory effects on steroid-receptor dependent gene expression of steroid-receptor dependent cancer cells in vitro and in vivo.

Sequences

Five tandem Gal4 DNA binding domain recognition sequences (SEQ ID NO:1, 103 nucleotides)

(SEQ ID NO: 1)
CGGAGTACTGTCCTCCGAGCGGAGTACTGTCCTCCGACTCGAGCGGAGTA
CTGTCCTCCGATCGGAGTACTGTCCTCCGCGAATTCCGGAGTACTGTCCT
CCG

Variants of Gal4 DNA binding domain recognition sequences may also be used.

Gal4 DNA binding domain coding sequence (SEQ ID NO:2, 441 nucleotides) (partial sequence of NCBI accession #AF264722)

(SEQ ID NO: 2)
ATGAAGCTACTGTCTTCTATCGAACAAGCATGCGATATTTGCCGACTTAA
AAAGCTCAAGTGCTCCAAAGAAAAACCGAAGTGCGCCAAGTGTCTGAAGA
ACAACTGGGAGTGTCGCTACTCTCCCAAAACCAAAAGGTCTCCGCTGACT
AGGGCACATCTGACAGAAGTGGAATCAAGGCTAGAAAGACTGGAACAGCT
ATTTCTACTGATTTTTCCTCGAGAAGACCTTGACATGATTTTGAAAATGG
ATTCTTTACAGGATATAAAAGCATTGTTAACAGGATTATTTGTACAAGAT
AATGTGAATAAAGATGCCGTCACAGATAGATTGGCTTCAGTGGAGACTGA
TATGCCTCTAACATTGAGACAGCATAGAATAAGTGCGACATCATCATCGG
AAGAGAGTAGTAACAAAGGTCAAAGACAGTTGACTGTATCG

Gal4 DNA binding domain (SEQ ID NO:3, 147 aa)

(SEQ ID NO: 3)
MKLLSSIEQACDICRLKKLKCSKEKPKCAKCLKNNWECRYSPKTKRSPLT
RAHLTEVESRLERLEQLFLLIFPREDLDMILKMDSLQDIKALLTGLFVQD
NVNKDAVTDRLASVETDMPLTLRQHRISATSSSEESSNKGQRQLTVS

Full ELK1 (ETS transcription factor (ELK1), transcript variant 1) protein coding sequence (partial sequence of ELK1 NCBI ACCESSION AB016193)

Nucleotide Sequence encoding full ELK1 protein (SEQ ID NO:4, 1,284 nucleotides)

(SEQ ID NO: 4)
ATGGACCCCAGCGTGACCCTGTGGCAGTTCCTGCTGCAGCTGCTGAGGGA
GCAGGGCAACGGCCACATCATCAGCTGGACCAGCAGGGACGGCGGCGAGT
TCAAGCTGGTGGACGCCGAGGAGGTGGCCAGGCTGTGGGGCCTGAGGAAG
AACAAGACCAACATGAACTACGACAAGCTGAGCAGGGCCCTGAGGTACTA
CTACGACAAGAACATCATCAGGAAGGTGAGCGGCCAGAAGTTCGTGTACA
AGTTCGTGAGCTACCCCGAGGTGGCCGGCTGCAGCACCGAGGACTGCCCC
CCCCAGCCCGAGGTGAGCGTGACCAGCACCATGCCCAACGTGGCCCCCGC
CGCCATCCACGCCGCCCCCGGCGACACCGTGAGCGGCAAGCCCGGCACCC
CCAAGGGCGCCGGCATGGCCGGCCCCGGCGGCCTGGCCAGGAGCAGCAGG
AACGAGTACATGAGGAGCGGCCTGTACAGCACCTTCACCATCCAGAGCCT
GCAGCCCCAGCCCCCCCCCCACCCCAGGCCCGCCGTGGTGCTGCCCAACG
CCGCCCCCGCCGGCGCCGCCGCCCCCCCCAGCGGCAGCAGGAGCACCAGC
CCCAGCCCCCTGGAGGCCTGCCTGGAGGCCGAGGAGGCCGGCCTGCCCCT
GCAGGTGATCCTGACCCCCCCCGAGGCCCCCAACCTGAAGAGCGAGGAGC
TGAACGTGGAGCCCGGCCTGGGCAGGGCCCTGCCCCCCGAGGTGAAGGTG
GAGGGCCCCAAGGAGGAGCTGGAGGTGGCCGGCGAGAGGGGCTTCGTGCC
CGAGACCACCAAGGCCGAGCCCGAGGTGCCCCCCCAGGAGGGCGTGCCCG
CCAGGCTGCCCGCCGTGGTGATGGACACCGCCGGCCAGGCCGGCGGCCAC
GCCGCCAGCAGCCCCGAGATCAGCCAGCCCCAGAAGGGCAGGAAGCCCAG
GGACCTGGAGCTGCCCCTGAGCCCCAGCCTGCTGGGCGGCCCCGGCCCCG
AGAGGACCCCCGGCAGCGGCAGCGGCAGCGGCCTGCAGGCCCCCGGCCCC
GCCCTGACCCCCAGCCTGCTGCCCACCCACACCCTGACCCCCGTGCTGCT
GACCCCCAGCAGCCTGCCCCCCAGCATCCACTTCTGGAGCACCCTGAGCC
CCATCGCCCCCAGGAGCCCCGCCAAGCTGAGCTTCCAGTTCCCCAGCAGC
GGCAGCGCCCAGGTGCACATCCCCAGCATCAGCGTGGACGGCCTGAGCAC
CCCCGTGGTGCTGAGCCCCGGCCCCCAGAAGCCC

Nucleotide sequence encoding ELK1 with deletion of first 86 amino acids compared to SEQ ID NO:4 (SEQ ID NO:5, 1,086 nucleotides)

(SEQ ID NO: 5)
AGCTACCCCGAGGTGGCCGGCTGCAGCACCGAGGACTGCCCCCCCCAGCC
CGAGGTGAGCGTGACCAGCACCATGCCCAACGTGGCCCCCGCCGCCATCC
ACGCCGCCCCCGGCGACACCGTGAGCGGCAAGCCCGGCACCCCCAAGGGC
GCCGGCATGGCCGGCCCCGGCGGCCTGGCCAGGAGCAGCAGGAACGAGTA
CATGAGGAGCGGCCTGTACAGCACCTTCACCATCCAGAGCCTGCAGCCCC
AGCCCCCCCCCCACCCCAGGCCCGCCGTGGTGCTGCCCAACGCCGCCCCC
GCCGGCGCCGCCGCCCCCCCCAGCGGCAGCAGGAGCACCAGCCCCAGCCC
CCTGGAGGCCTGCCTGGAGGCCGAGGAGGCCGGCCTGCCCCTGCAGGTGA
TCCTGACCCCCCCCGAGGCCCCCAACCTGAAGAGCGAGGAGCTGAACGTG
GAGCCCGGCCTGGGCAGGGCCCTGCCCCCCGAGGTGAAGGTGGAGGGCCC
CAAGGAGGAGCTGGAGGTGGCCGGCGAGAGGGGCTTCGTGCCCGAGACCA
CCAAGGCCGAGCCCGAGGTGCCCCCCCAGGAGGGCGTGCCCGCCAGGCTG
CCCGCCGTGGTGATGGACACCGCCGGCCAGGCCGGCGGCCACGCCGCCAG
CAGCCCCGAGATCAGCCAGCCCCAGAAGGGCAGGAAGCCCAGGGACCTGG
AGCTGCCCCTGAGCCCCAGCCTGCTGGGCGGCCCCGGCCCCGAGAGGACC
CCCGGCAGCGGCAGCGGCAGCGGCCTGCAGGCCCCCGGCCCCGCCCTGAC
CCCCAGCCTGCTGCCCACCCACACCCTGACCCCCGTGCTGCTGACCCCCA
GCAGCCTGCCCCCCAGCATCCACTTCTGGAGCACCCTGAGCCCCATCGCC
CCCAGGAGCCCCGCCAAGCTGAGCTTCCAGTTCCCCAGCAGCGGCAGCGC
CCAGGTGCACATCCCCAGCATCAGCGTGGACGGCCTGAGCACCCCCGTGG
TGCTGAGCCCCGGCCCCCAGAAGCCC

Complete ELK1 protein (SEQ ID NO:6, 428 aa)

(SEQ ID NO: 6)
MDPSVTLWQFLLQLLREQGSGHIISWTSRDGGEFKLVDAEEVARLWGLRK
NKTKMNYDKLSRALRYYYDKNIIRKVSGQKFVYKFVSYPEVAGCSTEDCP
PQPEVSVTSTMPNVAPAAIHAAPGDTVSGKPGTPKGAGMAGPGGLARSSR
NEYMRSGLYSTFTIQSLQPQPPPHPRPAVVLPNAAPAGAAAPPSGSRSTS
PSPLEACLEAEEAGLPLQVILTPPEAPNLKSEELNVEPGLGRALPPEVKV
EGPKEELEVAGERGFVPETTKAEPEVPPQEGVPARLPAVVMDTAGQAGGH
AASSPEISQPQKGRKPRDLELPLSPSLLGGPGPERTPGSGSGSGLQAPGP
ALTPSLLPTHTLTPVLLTPSSLPPSIHFWSTLSPIAPRSPAKLSFQFPSS
GSAQVHIPSISVDGLSTPWLSPGPQKP

ELK1 with deletion of first 86 amino acids compared to SEQ ID NO:6 (truncated ELK1, SEQ ID NO:7, 342 aa)

(SEQ ID NO: 7)
SYPEVAGCSTEDCPPQPEVSVTSTMPNVAPAAIHAAPGDTVSGKPGTPKG
AGMAGPGGLARSSRNEYMRSGLYSTFTIQSLQPQPPPHPRPAVVLPNAAP
AGAAAPPSGSRSTSPSPLEACLEAEEAGLPLQVILTPPEAPNLKSEELNV
EPGLGRALPPEVKVEGPKEELEVAGERGFVPETTKAEPEVPPQEGVPARL
PAVVMDTAGQAGGHAASSPEISQPQKGRKPRDLELPLSPSLLGGPGPERT
PGSGSGSGLQAPGPALTPSLLPTHTLTPVLLTPSSLPPSIHFWSTLSPIA
PRSPAKLSFQFPSSGSAQVHIPSISVDGLSTPVVLSPGPQKP

Amino acid sequence of the ELK1-Gal4 DNA binding domain fusion protein (SEQ ID NO:8, 489 aa)

(SEQ ID NO: 8)
MKLLSSIEQACDICRLKKLKCSKEKPKCAKCLKNNWECRYSPKTKRSPLT
RAHLTEVESRLERLEQLFLLIFPREDLDMILKMDSLQDIKALLTGLFVQD
NVNKDAVTDRLASVETDMPLTLRQHRISATSSSEESSNKGQRQLTVSSYP
EVAGCSTEDCPPQPEVSVTSTMPNVAPAAIHAAPGDTVSGKPGTPKGAGM
AGPGGLARSSRNEYMRSGLYSTFTIQSLQPQPPPHPRPAVVLPNAAPAGA
AAPPSGSRSTSPSPLEACLEAEEAGLPLQVILTPPEAPNLKSEELNVEPG
LGRALPPEVKVEGPKEELEVAGERGFVPETTKAEPEVPPQEGVPARLPAV
VMDTAGQAGGHAASSPEISQPQKGRKPRDLELPLSPSLLGGPGPERTPGS
GSGSGLQAPGPALTPSLLPTHTLTPVLLTPSSLPPSIHFWSTLSPIAPRS
PAKLSFQFPSSGSAQVHIPSISVDGLSTPVVLSPGPQKP

Variants, of truncated ELK1 (SEQ ID NO: 7) and Gal4 DNA binding domain (SEQ ID NO:3) can be used to produce variants of ELK1-Gal4 DNA binding domain fusion protein of SEQ ID NO:8 which may also be used.

Sequence Encoding Human Androgen Receptor (SEQ ID NO:9, 2,757 nucleotides) NCBI ACCESSION M20132 103180

(SEQ ID NO: 9)
ATGGAGGTGCAGCTGGGCCTGGGCAGGGTGTACCCCAGGCCCCCCAGCAA
GACCTACAGGGGCGCCTTCCAGAACCTGTTCCAGAGCGTGAGGGAGGTGA
TCCAGAACCCCGGCCCCAGGCACCCCGAGGCCGCCAGCGCCGCCCCCCCC
GGCGCCAGCCTGCTGCTGCTGCAGCAGCAGCAGCAGCAGCAGCAGCAGCA
GCAGCAGCAGCAGCAGCAGCAGCAGCAGCAGCAGGAGACCAGCCCCAGGC
AGCAGCAGCAGCAGCAGGGCGAGGACGGCAGCCCCCAGGCCCACAGGAGG
GGCCCCACCGGCTACCTGGTGCTGGACGAGGAGCAGCAGCCCAGCCAGCC
CCAGAGCGCCCTGGAGTGCCACCCCGAGAGGGGCTGCGTGCCCGAGCCCG
GCGCCGCCGTGGCCGCCAGCAAGGGCCTGOCCCAGCAGCTGCCCGCCCCC
CCCGACGAGGACGACAGCGCCGCCCCCAGCACCCTGAGCCTGCTGGGCCC
CACCTTCCCCGGCCTGAGCAGCTGCAGCGCCGACCTGAAGGACATCCTGA
GCGAGGCCAGCACCATGCAGCTGCTGCAGCAGCAGCAGCAGGAGGCCGTG
AGCGAGGGCAGCAGCAGCGGCAGGGCCAGGGAGGCCAGCGGCGCCCCCAC
CAGCAGCAAGGACAACTACCTGGGCGGCACCAGCACCATCAGCGACAACG
CCAAGGAGCTGTGCAAGGCCGTGAGCGTGAGCATGGGCCTGGGCGTGGAG
GCCCTGGAGCACCTGAGCCCCGGCGAGCAGCTGAGGGGCGACTGCATGTA
CGCCCCCCTGCTGGGCGTGCCCCCCGCCGTGAGGCCCACCCCCTGCGCCC
CCCTGGCCGAGTGCAAGGGCAGCCTGCTGGACGACAGCGCCGGCAAGAGC
ACCGAGGACACCGCCGAGTACAGCCCCTTCAAGGGCGGCTACACCAAGGG
CCTGGAGGGCGAGAGCCTGGGCTGCAGCGGCAGCGCCGCCGCCGGCAGCA
GCGGCACCCTGGAGCTGCCCAGCACCCTGAGCCTGTACAAGAGCGGCGCC
CTGGACGAGGCCGCCGCCTACCAGAGCAGGGACTACTACAACTTCCCCCT
GGCCCTGGCCGGCCCCCCCCCCCCCCCCCCCCCCCCCCACCCCCACGCCA
GGATCAAGCTGGAGAACCCCCTGGACTACGGCAGCGCCTGGGCCGCCGCC
GCCGCCCAGTGCAGGTACGGCGACCTGGCCAGCCTGCACGGCGCCGGCGC
CGCCGGCCCCGGCAGCGGCAGCCCCAGCGCCGCCGCCAGCAGCAGCTGGC
ACACCCTGTTCACCGCCGAGGAGGGCCAGCTGTACGGCCCCTGCGGCGGC
GGCGGCGGCGGCGGCGGCGGCGGCGGCGGCGGCGGCGGCGGCGGCGGCGG
CGGCGGCGGCGGCGGCGAGGCCGGCGCCGTGGCCCCCTACGGCTACACCA
GGCCCCCCCAGGGCCTGGCCGGCCAGGAGAGCGACTTCACCGCCCCCGAC
GTGTGGTACCCCGGCGGCATGGTGAGCAGGGTGCCCTACCCCAGCCCCAC
CTGCGTGAAGAGCGAGATGGGCCCCTGGATGGACAGCTACAGCGGCCCCT
ACGGCGACATGAGGCTGGAGACCGCCAGGGACCACGTGCTGCCCATCGAC
TACTACTTCCCCCCCCAGAAGACCTGCCTGATCTGCGGCGACGAGGCCAG
CGGCTGCCACTACGGCGCCCTGACCTGCGGCAGCTGCAAGGTGTTCTTCA
AGAGGGCCGCCGAGGGCAAGCAGAAGTACCTGTGCGCCAGCAGGAACGAC
TGCACCATCGACAAGTTCAGGAGGAAGAACTGCCCCAGCTGCAGGCTGAG
GAAGTGCTACGAGGCCGGCATGACCCTGGGCGCCAGGAAGCTGAAGAAGC
TGGGCAACCTGAAGCTGCAGGAGGAGGGCGAGGCCAGCAGCACCACCAGC
CCCACCGAGGAGACCACCCAGAAGCTGACCGTGAGCCACATCGAGGGCTA
CGAGTGCCAGCCCATCTTCCTGAACGTGCTGGAGGCCATCGAGCCCGGCG
TGGTGTGCGCCGGCCACGACAACAACCAGCCCGACAGCTTCGCCGCCCTG
CTGAGCAGCCTGAACGAGCTGGGCGAGAGGCAGCTGGTGCACGTGGTGAA
GTGGGCCAAGGCCCTGCCCGGCTTCAGGAACCTGCACGTGGACGACCAGA
TGGCCGTGATCCAGTACAGCTGGATGGGCCTGATGGTGTTCGCCATGGGC
TGGAGGAGCTTCACCAACGTGAACAGCAGGATGCTGTACTTCGCCCCCGA
CCTGGTGTTCAACGAGTACAGGATGCACAAGAGCAGGATGTACAGCCAGT
GCGTGAGGATGAGGCACCTGAGCCAGGAGTTCGGCTGGCTGCAGATCACC
CCCCAGGAGTTCCTGTGCATGAAGGCCCTGCTGCTGTTCAGCATCATCCC
CGTGGACGGCCTGAAGAACCAGAAGTTCTTCGACGAGCTGAGGATGAACT
ACATCAAGGAGCTGGACAGGATCATCGCCTGCAAGAGGAAGAACCCCACC
AGCTGCAGCAGGAGGTTCTACCAGCTGACCAAGCTGCTGGACAGCGTGCA
GCCCATCGCCAGGGAGCTGCACCAGTTCACCTTCGACCTGCTGATCAAGA
GCCACATGGTGAGCGTGGACTTCCCCGAGATGATGGCCGAGATCATCAGC
GTGCAGGTGCCCAAGATCCTGAGCGGCAAGGTGAAGCCCATCTACTTCCA
CACCCAG

Amino Acid Sequence of Human Androgen Receptor (919 aa)

(SEQ ID NO: 10)
MEVQLGLGRVYPRPPSKTYRGAFQNLFQSVREVIQNPGPRHPEAASAAPP
GASLLLLQQQQQQQQQQQQQQQQQQQQQETSPRQQQQQQGEDGSPQAHRR
GPTGYLVLDEEQQPSQPQSALECKPERGCVPEPGAAVAASKGLPQQLPAP
PDEDDSAAPSTLSLLGPTFPGLSSCSADLKDILSEASTMQLLQQQQQSAV
SEGSSSGRAREASGAPTSSKDNYLGGTSTISDNAKELCKAVSVSMGLGVE
ALEHLSPGEQLRGDCMYAPLLGVPPAVRPTPCAPLAECKGSLLDDSAGKS
TEDTAEYSPFKGGYTKGLEGESLGCSGSAAAGSSGTLELPSTLSLYKSGA
LDEAAAYQSRDYYNFPLALAGPPPPPPPPHPHARIKLENPLDYGSAWAAA
AAQCRYGDLASLHGAGAAGPGSGSPSAAASSSWHTLFTAEEGQLYGPCGG
GGGGGGGGGGGGGGGGGGGGGGEAGAVAPYGYTRPPQGLAGQESDFTAPD
VWYPGGKVSRVPYPSPTCVKSEMGPWMDSYSGPYGDMRLETARDHVLPID
YYFPPQKTCLICGDEASGCHYGALTCGSCKVFFKRAAEGKQKYLCASRND
CTIDKFRRKNCPSCRLRKCYEAGMTLGARKLKKLGNLKLQEEGEASSTTS
PTEETTQKLTVSHIEGYECQPIFLNVLEAIEPGVVCAGHDNNQPDSFAAL
LSSLNELGERQLVHVVKWAKALPGFRNLHVDDQMAVIQYSWMGLMVFAMG
WRSFTNVNSRMLYFAPDLVFKEYRMHKSRMYRQCVRMRHLRQEFGWLQLT
PQEFLCMKALLLFSIIPVDGLKNQKFFDELRMNYIKELDRIIACKRKNPT
SCSRRFYQLTKLLDSVQPIARELHQFTFDLLIKSHMVSVDFPEMMAEIIS
VQVPKILSGKVKPIYFHTQ

Variants, including splice variants, of human AR of SEQ ID NO:10 may also be used.

Canonical ARE sequence—SEQ ID NO:11

(SEQ ID NO: 11)
GCTTGTACAGGATGTTCTGCATGCTCTAGATGTACAGGATGTTCT

Variants of the canonical ARE (SEQ ID NO: 11) may also be used.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. An assay system for identifying a compound characterized by anti-hormone receptor-dependent cancer activity, comprising:
a first recombinant cell having a nucleus, the first recombinant cell comprising: 1) a reporter gene expression construct comprising a DNA binding domain recognition sequence in operable linkage with a nucleotide sequence encoding a reporter; 2) an expression construct encoding a fusion protein, the fusion protein comprising a DNA binding domain and ELK1 (ETS transcription factor (ELK1), transcript variant 1) lacking an ETS (E-twenty-six) DNA binding domain wherein the DNA binding domain is substituted for the ETS DNA binding domain of ELK1; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the first recombinant cell and specifically binds to ELK1 in the DNA binding domain-ELK1 fusion protein, thereby activating expression of the reporter gene in the first recombinant cell; and
a second recombinant cell, the second recombinant cell comprising: an androgen response element (ARE)-reporter gene expression construct comprising an ARE in operable linkage with a nucleic acid encoding a reporter, and an expression construct encoding human androgen receptor (AR), wherein in the presence of androgen, the AR enters the nucleus of the second recombinant cell and specifically binds to the ARE, thereby activating expression of the reporter gene in the second recombinant cell.

2. The assay system for identifying a compound characterized by anti-hormone receptor-dependent cancer activity according to claim 1, wherein the DNA binding domain recognition sequence of the reporter gene expression construct of the first recombinant cell comprises one or more contiguous Gal4 DNA binding domain recognition sequences; and wherein the DNA binding domain of the fusion protein is a Gal4 DNA binding domain that specifically binds to the one or more Gal4 DNA binding domain recognition sequences.

3. The assay system of claim 1, wherein one or more of the expression constructs selected from the group consisting of: the reporter gene expression construct; the expression construct encoding a fusion protein; the expression construct encoding AR, is stably integrated in the genome of the first recombinant cell.

4. The assay system of any of claim 1, wherein one or both of the expression constructs selected from the group consisting of: the ARE-reporter gene expression construct; and the expression construct encoding AR, is stably integrated in the genome of the second recombinant cell.

5. The assay system for identifying a compound characterized by anti-hormone receptor-dependent cancer activity of claim 1, wherein the reporter gene expression construct of the first recombinant cell and the reporter gene expression construct of the second recombinant cell both encode the same reporter.

6. The assay system for identifying a compound characterized by anti-hormone receptor-dependent cancer activity of claim 1, wherein the reporter gene expression construct of the first recombinant cell and the reporter gene expression construct of the second recombinant cell both encode firefly luciferase.

7. The assay system for identifying a compound characterized by anti-hormone receptor-dependent cancer activity of claim 1, wherein the reporter gene expression construct of the first recombinant cell and the reporter gene expression construct of the second recombinant cell encode different reporters.

8. An assay to identify a compound characterized by anti-hormone receptor-dependent cancer activity, comprising:
providing the first recombinant cell and the second recombinant cell of claim 1;
contacting the first recombinant cell and the second recombinant cell with a test compound; and
assaying the first recombinant cell and the second recombinant cell for expression of the reporter gene, wherein 1) partial or complete reversal of the activation of the reporter gene in the first recombinant cell in the presence of androgen and 2) no change in expression of the reporter gene in the second recombinant cell in the presence of androgen or a smaller reduction in reporter expression compared with the first recombinant cell, together indicate that the test compound inhibits interaction of the AR with ELK1, thereby identifying a compound characterized by anti-hormone receptor-dependent cancer activity.

9. The assay of claim 8 wherein the reporter gene expression construct of the first recombinant cell and the reporter gene expression construct of the second recombinant cell encode the same reporter and the first recombinant cell and the second recombinant cell are present in separate assay vessels.

10. The assay of claim 8 wherein the reporter gene expression construct of the first recombinant cell and the reporter gene expression construct of the second recombinant cell encode different reporters and the first recombinant cell and the second recombinant cell are present in the same assay vessel.

11. A recombinant cell having a nucleus, the first recombinant cell comprising: 1) a reporter gene expression construct comprising a DNA binding domain recognition sequence in operable linkage with a nucleotide sequence encoding a reporter, 2) an expression construct encoding a fusion protein, the fusion protein comprising a DNA binding domain and ELK1 lacking an ETS DNA binding domain wherein the DNA binding domain is substituted for the ETS DNA binding domain of ELK1; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the first recombinant cell and specifically binds to ELK1 in the DNA binding domain-ELK1 fusion protein, thereby activating expression of the reporter gene in the first recombinant cell.

12. The recombinant cell according to claim 11, wherein the DNA binding domain recognition sequence of the reporter gene expression construct of the first recombinant cell comprises one or more contiguous Gal4 DNA binding domain recognition sequences; and wherein the wherein the DNA binding domain of the fusion protein is a Gal4 DNA binding domain that specifically binds to the one or more Gal4 DNA binding domain recognition sequences.

13. The recombinant cell according to claim 11, wherein the reporter gene encodes firefly luciferase.

14. The recombinant cell of claim 11, wherein one or more of the expression constructs selected from the group consisting of: the reporter gene expression construct; the expression construct encoding a fusion protein; the expression construct encoding AR, is stably integrated in the genome of the recombinant cell.

15. An assay system for identifying a compound characterized by anti-hormone receptor-dependent cancer activity, comprising:
a recombinant cell having a nucleus, the recombinant cell comprising: 1) a first reporter gene expression construct comprising a DNA binding domain recognition sequence in operable linkage with a nucleotide sequence encoding a first reporter; 2) an expression construct encoding a fusion protein, the fusion protein comprising a DNA binding domain and ELK1 lacking an ETS DNA binding domain wherein the DNA binding domain is substituted for the ETS DNA binding domain of ELK1; and 3) an expression construct encoding human androgen receptor (AR); wherein the DNA binding domain of the DNA binding domain-ELK1 fusion protein specifically binds to the DNA binding domain recognition sequence of the reporter gene expression construct, and wherein, in the presence of androgen, the AR enters the nucleus of the recombinant cell and specifically binds to ELK1 in the DNA binding domain-ELK1 fusion protein, thereby activating expression of the first reporter gene in the recombinant cell; and
the recombinant cell further comprising: an androgen response element (ARE)-reporter gene expression construct comprising an ARE in operable linkage with a nucleic acid encoding a second reporter; and an expression construct encoding human androgen receptor (AR), wherein in the presence of androgen, the AR enters the nucleus of the recombinant cell and specifically binds to the ARE, thereby activating expression of the second reporter gene in the recombinant cell, wherein the first and second reporter genes express detectably different reporters.

16. The assay system for identifying a compound characterized by anti-hormone receptor-dependent cancer activity according to claim 15, wherein the DNA binding domain recognition sequence of the reporter gene expression construct of the recombinant cell comprises one or more contiguous Gal4 DNA binding domain recognition sequences; and wherein the DNA binding domain of the fusion protein is a Gal4 DNA binding domain that specifically binds to the one or more Gal4 DNA binding domain recognition sequences.

17. The assay system of claim 15, wherein one or more of the expression constructs selected from the group consisting of: the first reporter gene expression construct; the expression construct encoding a fusion protein; the expression construct encoding AR, the ARE-reporter gene expression construct; and the expression construct encoding AR, is stably integrated in the genome of the recombinant cell.

18. An assay to identify a compound characterized by anti-hormone receptor-dependent cancer activity, comprising:
providing the recombinant cell of claim 15;
contacting the recombinant cell with a test compound; and
assaying the recombinant cell for expression of the reporter gene, wherein 1) partial or complete reversal of the activation of the reporter gene in the first recombinant cell in the presence of androgen and 2) no change in expression of the reporter gene in the second recombinant cell in the presence of androgen or a smaller reduction in reporter expression compared with the first recombinant cell, together indicate that the test compound inhibits interaction of the AR with ELK1, thereby identifying a compound characterized by anti-hormone receptor-dependent cancer activity.


SEQUENCE LISTING (11 sequences)

SEQ ID NO: 1 - DNA, 103 bp, Artificial Sequence
  Five tandem Gal4 DNA binding domain recognition sequences
cggagtactgtcctccgagcggagtactgtcctccgactcgagcggagtactgtcctccgatcggagtactgtcctccgcgaattccggagtactgtcctccg

SEQ ID NO: 2 - DNA, 441 bp, Artificial Sequence
  Gal4 DNA binding domain coding sequence
atgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcg

SEQ ID NO: 3 - protein, 147 aa, Artificial Sequence
  Gal4 DNA binding domain
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVS

SEQ ID NO: 4 - DNA, 1284 bp, Homo sapiens
atggaccccagcgtgaccctgtggcagttcctgctgcagctgctgagggagcagggcaacggccacatcatcagctggaccagcagggacggcggcgagttcaagctggtggacgccgaggaggtggccaggctgtggggcctgaggaagaacaagaccaacatgaactacgacaagctgagcagggccctgaggtactactacgacaagaacatcatcaggaaggtgagcggccagaagttcgtgtacaagttcgtgagctaccccgaggtggccggctgcagcaccgaggactgccccccccagcccgaggtgagcgtgaccagcaccatgcccaacgtggcccccgccgccatccacgccgcccccggcgacaccgtgagcggcaagcccggcacccccaagggcgccggcatggccggccccggcggcctggccaggagcagcaggaacgagtacatgaggagcggcctgtacagcaccttcaccatccagagcctgcagccccagccccccccccaccccaggcccgccgtggtgctgcccaacgccgcccccgccggcgccgccgccccccccagcggcagcaggagcaccagccccagccccctggaggcctgcctggaggccgaggaggccggcctgcccctgcaggtgatcctgaccccccccgaggcccccaacctgaagagcgaggagctgaacgtggagcccggcctgggcagggccctgccccccgaggtgaaggtggagggccccaaggaggagctggaggtggccggcgagaggggcttcgtgcccgagaccaccaaggccgagcccgaggtgcccccccaggagggcgtgcccgccaggctgcccgccgtggtgatggacaccgccggccaggccggcggccacgccgccagcagccccgagatcagccagccccagaagggcaggaagcccagggacctggagctgcccctgagccccagcctgctgggcggccccggccccgagaggacccccggcagcggcagcggcagcggcctgcaggcccccggccccgccctgacccccagcctgctgcccacccacaccctgacccccgtgctgctgacccccagcagcctgccccccagcatccacttctggagcaccctgagccccatcgcccccaggagccccgccaagctgagcttccagttccccagcagcggcagcgcccaggtgcacatccccagcatcagcgtggacggcctgagcacccccgtggtgctgagccccggcccccagaagccc

SEQ ID NO: 5 - DNA, 1026 bp, Artificial Sequence
  Nucleotide sequence encoding ELK1 with deletion of first 86 amino acids compared to SEQ ID NO4
agctaccccgaggtggccggctgcagcaccgaggactgccccccccagcccgaggtgagcgtgaccagcaccatgcccaacgtggcccccgccgccatccacgccgcccccggcgacaccgtgagcggcaagcccggcacccccaagggcgccggcatggccggccccggcggcctggccaggagcagcaggaacgagtacatgaggagcggcctgtacagcaccttcaccatccagagcctgcagccccagccccccccccaccccaggcccgccgtggtgctgcccaacgccgcccccgccggcgccgccgccccccccagcggcagcaggagcaccagccccagccccctggaggcctgcctggaggccgaggaggccggcctgcccctgcaggtgatcctgaccccccccgaggcccccaacctgaagagcgaggagctgaacgtggagcccggcctgggcagggccctgccccccgaggtgaaggtggagggccccaaggaggagctggaggtggccggcgagaggggcttcgtgcccgagaccaccaaggccgagcccgaggtgcccccccaggagggcgtgcccgccaggctgcccgccgtggtgatggacaccgccggccaggccggcggccacgccgccagcagccccgagatcagccagccccagaagggcaggaagcccagggacctggagctgcccctgagccccagcctgctgggcggccccggccccgagaggacccccggcagcggcagcggcagcggcctgcaggcccccggccccgccctgacccccagcctgctgcccacccacaccctgacccccgtgctgctgacccccagcagcctgccccccagcatccacttctggagcaccctgagccccatcgcccccaggagccccgccaagctgagcttccagttccccagcagcggcagcgcccaggtgcacatccccagcatcagcgtggacggcctgagcacccccgtggtgctgagccccggcccccagaagccc

SEQ ID NO: 6 - protein, 428 aa, Homo sapiens
MDPSVTLWQFLLQLLREQGNGHIISWTSRDGGEFKLVDAEEVARLWGLRKNKTNMNYDKLSRALRYYYDKNIIRKVSGQKFVYKFVSYPEVAGCSTEDCPPQPEVSVTSTMPNVAPAAIHAAPGDTVSGKPGTPKGAGMAGPGGLARSSRNEYMRSGLYSTFTIQSLQPQPPPHPRPAVVLPNAAPAGAAAPPSGSRSTSPSPLEACLEAEEAGLPLQVILTPPEAPNLKSEELNVEPGLGRALPPEVKVEGPKEELEVAGERGFVPETTKAEPEVPPQEGVPARLPAVVMDTAGQAGGHAASSPEISQPQKGRKPRDLELPLSPSLLGGPGPERTPGSGSGSGLQAPGPALTPSLLPTHTLTPVLLTPSSLPPSIHFWSTLSPIAPRSPAKLSFQFPSSGSAQVHIPSISVDGLSTPVVLSPGPQKP

SEQ ID NO: 7 - protein, 342 aa, Artificial Sequence
  ELK1 with deletion of first 86 amino acids compared to SEQ ID NO6 (truncated ELK1)
SYPEVAGCSTEDCPPQPEVSVTSTMPNVAPAAIHAAPGDTVSGKPGTPKGAGMAGPGGLARSSRNEYMRSGLYSTFTIQSLQPQPPPHPRPAVVLPNAAPAGAAAPPSGSRSTSPSPLEACLEAEEAGLPLQVILTPPEAPNLKSEELNVEPGLGRALPPEVKVEGPKEELEVAGERGFVPETTKAEPEVPPQEGVPARLPAVVMDTAGQAGGHAASSPEISQPQKGRKPRDLELPLSPSLLGGPGPERTPGSGSGSGLQAPGPALTPSLLPTHTLTPVLLTPSSLPPSIHFWSTLSPIAPRSPAKLSFQFPSSGSAQVHIPSISVDGLSTPVVLSPGPQKP

SEQ ID NO: 8 - protein, 489 aa, Artificial Sequence
  ELK1-Gal4 DNA binding domain fusion protein
MKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSSYPEVAGCSTEDCPPQPEVSVTSTMPNVAPAAIHAAPGDTVSGKPGTPKGAGMAGPGGLARSSRNEYMRSGLYSTFTIQSLQPQPPPHPRPAVVLPNAAPAGAAAPPSGSRSTSPSPLEACLEAEEAGLPLQVILTPPEAPNLKSEELNVEPGLGRALPPEVKVEGPKEELEVAGERGFVPETTKAEPEVPPQEGVPARLPAVVMDTAGQAGGHAASSPEISQPQKGRKPRDLELPLSPSLLGGPGPERTPGSGSGSGLQAPGPALTPSLLPTHTLTPVLLTPSSLPPSIHFWSTLSPIAPRSPAKLSFQFPSSGSAQVHIPSISVDGLSTPVVLSPGPQKP

SEQ ID NO: 9 - DNA, 2757 bp, Homo sapiens
atggaggtgcagctgggcctgggcagggtgtaccccaggccccccagcaagacctacaggggcgccttccagaacctgttccagagcgtgagggaggtgatccagaaccccggccccaggcaccccgaggccgccagcgccgccccccccggcgccagcctgctgctgctgcagcagcagcagcagcagcagcagcagcagcagcagcagcagcagcagcagcagcagcagcaggagaccagccccaggcagcagcagcagcagcagggcgaggacggcagcccccaggcccacaggaggggccccaccggctacctggtgctggacgaggagcagcagcccagccagccccagagcgccctggagtgccaccccgagaggggctgcgtgcccgagcccggcgccgccgtggccgccagcaagggcctgccccagcagctgcccgccccccccgacgaggacgacagcgccgcccccagcaccctgagcctgctgggccccaccttccccggcctgagcagctgcagcgccgacctgaaggacatcctgagcgaggccagcaccatgcagctgctgcagcagcagcagcaggaggccgtgagcgagggcagcagcagcggcagggccagggaggccagcggcgcccccaccagcagcaaggacaactacctgggcggcaccagcaccatcagcgacaacgccaaggagctgtgcaaggccgtgagcgtgagcatgggcctgggcgtggaggccctggagcacctgagccccggcgagcagctgaggggcgactgcatgtacgcccccctgctgggcgtgccccccgccgtgaggcccaccccctgcgcccccctggccgagtgcaagggcagcctgctggacgacagcgccggcaagagcaccgaggacaccgccgagtacagccccttcaagggcggctacaccaagggcctggagggcgagagcctgggctgcagcggcagcgccgccgccggcagcagcggcaccctggagctgcccagcaccctgagcctgtacaagagcggcgccctggacgaggccgccgcctaccagagcagggactactacaacttccccctggccctggccggccccccccccccccccccccccccccacccccacgccaggatcaagctggagaaccccctggactacggcagcgcctgggccgccgccgccgcccagtgcaggtacggcgacctggccagcctgcacggcgccggcgccgccggccccggcagcggcagccccagcgccgccgccagcagcagctggcacaccctgttcaccgccgaggagggccagctgtacggcccctgcggcggcggcggcggcggcggcggcggcggcggcggcggcggcggcggcggcggcggcggcggcggcggcggcgaggccggcgccgtggccccctacggctacaccaggcccccccagggcctggccggccaggagagcgacttcaccgcccccgacgtgtggtaccccggcggcatggtgagcagggtgccctaccccagccccacctgcgtgaagagcgagatgggcccctggatggacagctacagcggcccctacggcgacatgaggctggagaccgccagggaccacgtgctgcccatcgactactacttccccccccagaagacctgcctgatctgcggcgacgaggccagcggctgccactacggcgccctgacctgcggcagctgcaaggtgttcttcaagagggccgccgagggcaagcagaagtacctgtgcgccagcaggaacgactgcaccatcgacaagttcaggaggaagaactgccccagctgcaggctgaggaagtgctacgaggccggcatgaccctgggcgccaggaagctgaagaagctgggcaacctgaagctgcaggaggagggcgaggccagcagcaccaccagccccaccgaggagaccacccagaagctgaccgtgagccacatcgagggctacgagtgccagcccatcttcctgaacgtgctggaggccatcgagcccggcgtggtgtgcgccggccacgacaacaaccagcccgacagcttcgccgccctgctgagcagcctgaacgagctgggcgagaggcagctggtgcacgtggtgaagtgggccaaggccctgcccggcttcaggaacctgcacgtggacgaccagatggccgtgatccagtacagctggatgggcctgatggtgttcgccatgggctggaggagcttcaccaacgtgaacagcaggatgctgtacttcgcccccgacctggtgttcaacgagtacaggatgcacaagagcaggatgtacagccagtgcgtgaggatgaggcacctgagccaggagttcggctggctgcagatcaccccccaggagttcctgtgcatgaaggccctgctgctgttcagcatcatccccgtggacggcctgaagaaccagaagttcttcgacgagctgaggatgaactacatcaaggagctggacaggatcatcgcctgcaagaggaagaaccccaccagctgcagcaggaggttctaccagctgaccaagctgctggacagcgtgcagcccatcgccagggagctgcaccagttcaccttcgacctgctgatcaagagccacatggtgagcgtggacttccccgagatgatggccgagatcatcagcgtgcaggtgcccaagatcctgagcggcaaggtgaagcccatctacttccacacccag

SEQ ID NO: 10 - protein, 919 aa, Homo sapiens
MEVQLGLGRVYPRPPSKTYRGAFQNLFQSVREVIQNPGPRHPEAASAAPPGASLLLLQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 11 - DNA, 45 bp, Artificial Sequence
  Canonical Androgen Response Element (ARE) sequence
gcttgtacaggatgttctgcatgctctagatgtacaggatgttct